(19) United States
(12) Patent Application Publication
(10) Pub. No.: US 2006/0191079 A1
(43) Pub. Date: Aug. 31, 2006

(54) Composition for bleaching and simultaneously dyeing keratin fibers, comprising quinoline or a quinoline derivative

(76) Inventors: Sylvain Kravtchenko, Asnieres (FR); Valerie Bonnardel, Colombes (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/293,264

(22) Filed: Dec. 5, 2005

Related U.S. Application Data
(60) Provisional application No. 60/670,673, filed on Apr. 13, 2005.

(30) Foreign Application Priority Data
Dec. 3, 2004 (FR) 04 52858

(51) Int. Cl. A61K 8/00

(52) U.S. Cl. 8/405

(57) ABSTRACT

The present invention relates to a composition for bleaching and simultaneously dyeing keratin fibers, comprising at least one dye chosen from quinoline derivatives and addition salts thereof, at least one peroxygenated salt and at least one alkaline agent, to the process for bleaching and dyeing keratin fibers using this composition, and also to the use of this composition for bleaching and simultaneously dyeing keratin fibers. The composition in accordance with the present invention is particularly suitable for dark hair. It has improved stability over time and allows chromatic and/or fast dyeing to be obtained.

This application claims benefit of U.S. Provisional Application No. 60/670,673, filed Apr. 13, 2005, the contents of which are incorporated herein by reference. This application also claims benefit of priority under 35 U.S.C. § 119 to French Patent Application No. 04 52858, filed Dec. 3, 2004, the contents of which are also incorporated herein by reference.

The present disclosure relates to a composition for bleaching and simultaneously dyeing keratin fibers, for example, human keratin fibers such as the hair, comprising at least one dye chosen from quinoline, quinoline derivatives and addition salts thereof, at least one peroxygenated salt and at least one alkaline agent.

When a person wishes to radically change the color of his or her hair, especially when he or she wishes to obtain a color lighter than his or her original color, it is often necessary to bleach and, where necessary, to dye the hair. Several methods exist for doing this.

The first method comprises using lightening products based on aqueous ammonia and hydrogen peroxide. These products may optionally contain dyes, which allows the hair to be lightened and simultaneously dyed. However, the lightening performance of these products remains limited, especially for applications to natural and/or dyed dark foundation colors.

The second method comprises applying to the hair a lightening composition based on peroxygenated salts such as persulfate and alkaline agents to which has been added hydrogen peroxide at the time of use, in order to obtain greater lightening. This type of product is typically very satisfactory and more suited to dark foundation color, but leads to only a very restricted range of tints. It may thus be necessary to correct the shade obtained by applying in a second stage a dye product to the hair. This two-step process has the drawback of being relatively long.

To overcome this drawback, it is known practice to add dyes to these lightening products. This method allows the hair fiber to be dyed and simultaneously bleached. Since the level of lightening is substantial, it is suited, for example, to natural and/or dyed foundation colors. However, there is a very limited number of dyes that are stable under these highly oxidative conditions, which limits the variety of tints that may be obtained. Moreover, this instability is reflected by a more or less rapid change in the tint during application, which may lead to poorly reproducible results.

Moreover, the fastness of these dyes with respect to external agents, such as light and shampoo, is not always satisfactory.

Direct dyes of anthraquinone, azo, triarylmethane, thiazine, quinone and nitro type, which are stable in these highly oxidative media, have been proposed in U.S. Pat. No. 5,688,291, International Published Application No. WO 02/074 270 and German utility model patent DE 203 03 559. However, these dyes may be unsatisfactory in terms of chromaticity, fastness and/or stability during the application time.

Thus it would be desirable to provide novel compositions for bleaching and simultaneously dyeing keratin fibers, for example human keratin fibers such as the hair, which may be suitable for dark foundation colors, which show good stability over time and/or which allow chromatic and fast colorations to be obtained.

At least one of these advantages is achieved with the present invention, which relates to, among other things, a composition for bleaching and simultaneously dyeing keratin fibers, comprising:

at least one dye chosen from quinoline, quinoline derivatives and the addition salts thereof;

at least one peroxygenated salt; and

at least one alkaline agent.

The composition in accordance with the present disclosure is useful, for example, for bleaching and simultaneously dyeing dark hair. It shows improved stability over time and/or allows a chromatic coloration to be obtained. Furthermore, with suitable concentrations of dyes according to the disclosure, pastel tints may be obtained.

This coloration is resistant to at least one of the various attacking factors to which hair may be subjected, such as shampoo, rubbing, light, bad weather, sweat and permanent reshaping operations. It is also powerful, aesthetic and/or, furthermore, sparingly selective, i.e., it may produce only small differences between different parts of a hair or of a head of hair that are differently sensitized.

The present disclosure also relates to a process for bleaching and simultaneously dyeing keratin fibers, using the composition in accordance with the present disclosure, and also multi-compartment devices for implementing this process.

Another subject of the present disclosure is the use of the composition in accordance with the present disclosure for bleaching and simultaneously dyeing keratin fibers.

According to one embodiment of the present disclosure, the at least one dye is chosen from the compounds of formula (I) and (I′) below, and the addition salts thereof: 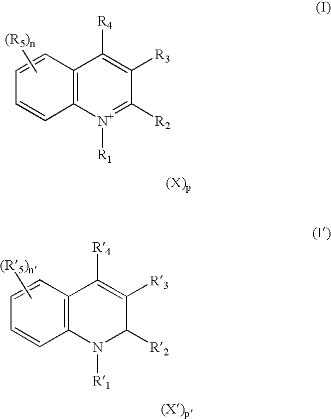
in which:

R₁ and R₄ are chosen, independently of each other, from:

a hydrogen atom;

alkyl radicals; and

hydroxyalkyl radicals;

R₂ is chosen from:

a hydrogen atom; and

an alkyl radical;

R₃ is chosen from:

a hydrogen atom;

a saturated or unsaturated, 5- or 6-membered heterocyclic radical optionally fused with at least one ring chosen from aromatic or non-aromatic, heterocyclic or non-heterocyclic rings, the whole group comprising from 3 to 20 carbon atoms and from 1 to 4 hetero atoms, which may be substituted or unsubstituted, and optionally bearing a cationic charge; and

an alkyl radical;

R₅ is chosen from:

an alkyl radical;

an amino radical;

a monoalkylamino or dialkylamino radical;

a mono(hydroxyalkyl)amino or di(hydroxyalkyl)amino radical;

an N,N-(alkyl)(hydroxyalkyl)amino radical; and

a sulfonato radical;

n is an integer ranging from 0 to 4, with the proviso that when n is greater than or equal to 2, then the radicals R₅ may be identical or different;

X denotes a negatively charged organic or mineral atom or group of atoms to ensure the overall neutrality of the molecule;

p is an integer equal to 0 or 1;

R′₁, R′₃, R′₄, R′₅, n′, X′ and p′ have, respectively, the same definition as R₁, R₃, R₄, R₅, n, X and p;

R′₂ is chosen from:

a hydrogen atom;

an alkyl radical;

a disubstituted methylidene radical of formula (II): 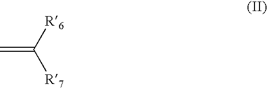
in which R′₆ and R′₇ are chosen from, independently of each other, alkyl radicals; and hydroxyalkyl radicals; or together form, with the carbon atom to which they are attached, a saturated or unsaturated 5- or 6-membered ring in which at least one carbon atom may be replaced with a carbonyl group, optionally fused with at least one aromatic or non-aromatic, heterocyclic or non-heterocyclic ring, the whole group possibly being unsubstituted or substituted with at least one radical chosen from an alkyl radical, an alkoxy radical and an amino radical.

In the context of the present disclosure, the term “alkyl radical” (alk) means a linear or branched radical containing from 1 to 6 carbon atoms, for example a methyl, ethyl, n-propyl, isopropyl, n-butyl or tert-butyl radical. An alkoxy radical is a radical alk-O—, a monoalkylamino or dialkylamino radical is a radical (alk)ₙN— with n=1 or 2, the alkyl radical being as defined above.

A substituted alkyl radical is a monosubstituted or polysubstituted alkyl radical. For example, a hydroxyalkyl radical is an alkyl radical that may be substituted with at least one hydroxyl group, and a haloalkyl radical is an alkyl radical that may be substituted with at least one halo group.

A saturated or unsaturated 5- or 6-membered heterocyclic radical optionally fused with at least one aromatic or non-aromatic, heterocyclic or non-heterocyclic ring, the whole group containing from 3 to 20 carbon atoms and from 1 to 4 hetero atoms and optionally bearing a cationic charge, may be, for example, a quinolinium radical or an indolium radical.

A saturated or unsaturated 5- or 6-membered ring in which at least one carbon atom may be replaced with a carbonyl group, optionally fused with at least one aromatic or non-aromatic, heterocyclic or non-heterocyclic ring, may be, for example, an indanedione radical.

In all the above meanings, and unless otherwise indicated, when a group is substituted, it is monosubstituted or polysubstituted and the substituents are chosen from halo, hydroxyl, alkyl, hydroxyalkyl, haloalkyl, alkoxy, amino, mono(alkyl)amino or di(alkyl)-amino, mono(hydroxyalkyl)amino or di(hydroxyalkyl)amino and carboxyl radicals.

According to one embodiment of the present disclosure, R. and R₄ are chosen from, independently of each other, a hydrogen atom and alkyl radicals. By way of example, R₁ and R₄ are chosen from a hydrogen atom; a methyl radical; and an ethyl radical.

According to one embodiment of the present disclosure, R₂ is chosen from a hydrogen atom and an alkyl radical. By way of example, R₂ is chosen from a hydrogen atom and a methyl radical.

According to one embodiment of the present disclosure, R₃ is chosen from a hydrogen atom; a substituted or unsubstituted quinolinium radical; and a substituted or unsubstituted 3H-indolium radical. By way of example, R₃ is chosen from a hydrogen atom; a 1-methylquinolinium radical; and a 1,3,3′-trimethyl-3H-indolium radical.

According to one embodiment of the present disclosure, R₅ is chosen from a mono(alkyl)amino radical and a di(alkyl)amino radical. By way of example, R₅ is a dimethylamino radical.

According to one embodiment of the present disclosure, n is equal to 0 or 1.

According to one embodiment of the present disclosure, R′₁ and R′₄ are both a hydrogen atom.

According to one embodiment of the present disclosure, R′₂ is a disubstituted methylidene radical of formula (II). By way of example, R′₂ is an indanedione radical.

According to one embodiment of the present disclosure, R′₃ is a hydrogen atom.

According to one embodiment of the present disclosure, R′₅ is chosen from a sulfonato radical and an alkyl radical. By way of example, R₅ is chosen from a sulfonato radical and a methyl radical.

According to one embodiment of the present disclosure, n′ ranges from 1 to 4.

According to one embodiment of the present disclosure, at least one of the radicals R′₅ is a sulfonato radical. For example, at least one of the radicals R′₅ may be a sulfonato radical in sodium salt form.

As examples of dyes that are useful in the context of the present disclosure, mention may be made of the following compounds: 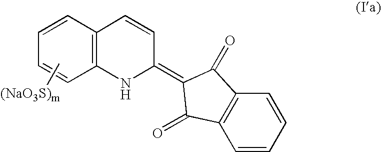
in which m is an integer ranging from 1 to 3; 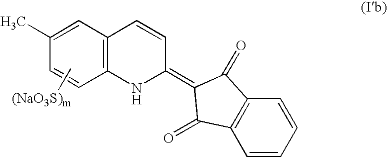
in which m is an integer ranging from 1 to 3;

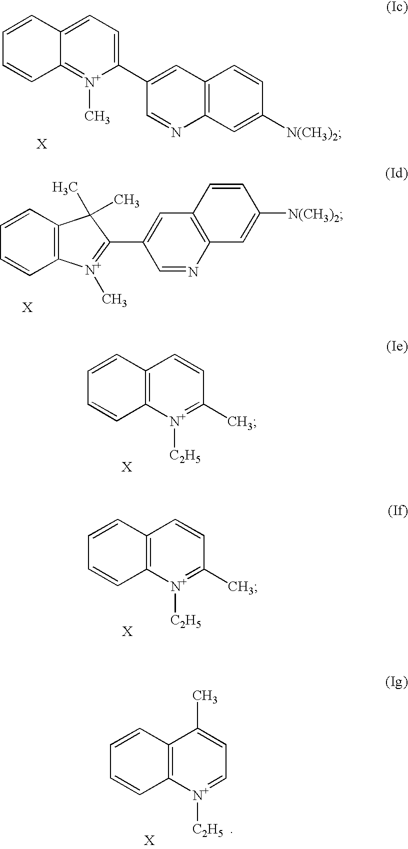

According to one embodiment of the present disclosure, X is chosen from an alkylsulfonate and a halide. By way of example, X is chosen from a methylsulfonate; a chloride; and an iodide.

According to one embodiment of the present disclosure, the at least one dye disclosed herein is chosen from the compounds of formula (I′), and the addition salts thereof.

According to one embodiment of the present disclosure, the at least one dye is chosen from the compounds of formula (I′a); the compounds of formula (I′b); and the addition salts thereof.

For example, the dye Food Yellow 13 is useful in the context of the present disclosure.

In general, the addition salts of the quinoline derivatives that may be used in the context of the present disclosure are chosen from the addition salts with an organic or mineral base, and, for example the salts of alkali metals or of alkaline-earth metals and the salts of organic amines such as alkanolamines. In at least one embodiment, sodium salts are used.

The concentration of quinoline and/or quinoline derivatives and/or addition salts thereof in the composition in accordance with the present disclosure generally ranges from 0.0001% to 10% by weight, such as from 0.001% to 8% and, for example, from 0.01% to 5% by weight, relative to the total weight of the composition.

The at least one peroxygenated salt that is useful in the present disclosure is chosen, for example, from alkali metal or alkaline-earth metal persulfates, perborates, percarbonates and peroxides, and mixtures thereof. Persulfates and mixtures thereof, and, for instance, such as sodium persulfate, potassium persulfate and ammonium persulfate, and mixtures thereof, are used in at least one embodiment.

The concentration of peroxygenated salts in the composition in accordance with the present disclosure generally ranges from 10% to 70% by weight and, for example, from 20% and 60% by weight, relative to the total weight of the composition.

The at least one alkaline agent that is useful in the composition of the present disclosure is chosen, for example, from urea, ammonium salts, for instance ammonium chloride, ammonium sulfate, ammonium phosphate or ammonium nitrate, silicates, phosphates or carbonates of alkali metals or of alkaline-earth metals such as lithium, sodium, potassium, magnesium, calcium or barium, and mixtures thereof. In at least one embodiment, the at least alkaline agent is chosen from ammonium chloride, silicates and carbonates, and mixtures thereof.

The concentration of alkaline agents in the composition in accordance with the present disclosure generally ranges from 0.01% to 40% by weight, such as from 0.1% to 30% by weight, relative to the total weight of the composition.

The composition in accordance with the present disclosure may be in the form of a powder or a paste. In at least one embodiment, the composition is in the form of a paste.

When the composition in accordance with the present disclosure is in the form of a paste, it also comprises at least one inert organic liquid phase.

For the purposes of the present disclosure, the term “liquid phase” means any phase capable of flowing at room temperature, generally from 15° C. to 40° C., and at atmospheric pressure, under the action of its own weight.

Examples of inert liquid phases that may be mentioned include the polydecenes of formula C₁₀ₙH_{[(20n)+2]} in which n ranges from 3 to 9, such as from 3 to 7, esters of fatty alcohols or of fatty acids, C₁₂-C₂₄ fatty acid esters or diesters of sugars, cyclic ethers or cyclic esters, silicone oils, mineral oils and plant oils, or mixtures thereof.

The compounds of formula C₁₀ₙH_{[(20n)+2]} with n ranging from 3 to 9 correspond to the name “polydecene” of the CTFA dictionary 7th edition, 1997 of the Cosmetic, Toiletry and Fragrance Association, USA, and also to the same INCI name in USA and Europe. These are products of hydrogenation of poly-1-decenes.

Among these compounds, the ones that may be useful in at least one embodiment of the present disclosure are those for which, in the formula, n ranges from 3 to 7.

Examples that may be mentioned include the product sold under the name Silkflo® 366 NF Polydecene by the company Amoco Chemical, and those sold under the name Nexbase® 2002 FG, 2004 FG, 2006 FG and 2008 FG by the company Fortum.

As regards the esters of fatty alcohols or of fatty acids, examples that may be mentioned include:
esters of linear or branched, saturated lower C₃-C₆ monoalcohols with C₁₂-C₂₄ monofunctional fatty acids, these fatty acids possibly being linear or branched, and saturated or unsaturated, and chosen, for example, from oleates, laurates, palmitates, myristates, behenates, cocoates, stearates, linoleates, linolenates, caprates and arachidonates, or mixtures thereof, for example, such as oleo-palmitates, oleostearates and palmitostearates. In at least one embodiment, at least one ester chosen from isopropyl palmitate, isopropyl myristate and octyldodecyl stearate may be used;
esters of linear or branched C₃-C₈ monoalcohols with C₈-C₂₄ difunctional fatty acids, these fatty acids possibly being linear or branched, and saturated or unsaturated, for instance the isopropyl diester of sebacic acid, also known as diisopropyl sebacate;
esters of linear or branched C₃-C₈ monoalcohols with C₂-C₈ difunctional fatty acids, these fatty acids possibly being linear or branched, and saturated or unsaturated, for instance dioctyl adipate and dicaprylyl maleate;
the ester of a trifunctional acid, for instance triethyl citrate.

As regards the C₁₂-C₂₄ fatty acid esters and diesters of sugars, the term “sugar” as used herein means compounds containing several alcohol functions, with or without an aldehyde or ketone function, and which contain at least four carbon atoms. These sugars may be monosaccharides, oligosaccharides or polysaccharides.

As sugars that may be used according to the present disclosure, mention may be made, for example, of sucrose (or saccharose), glucose, galactose, ribose, fucose, maltose, fructose, mannose, arabinose, xylose and lactose, and derivatives thereof, for example, alkyl derivatives, such as methyl derivatives, for instance methylglucose.

The fatty acid esters of sugars that may be used according to the present disclosure may be chosen from the group comprising esters or mixtures of esters of sugars described above and of linear or branched, saturated or unsaturated C₁₂-C₂₄ fatty acids.

The esters may be chosen from mono-, di-, tri-, tetraesters and polyesters, and mixtures thereof.

These esters may be chosen, for example, from oleates, laurates, palmitates, myristates, behenates, cocoates, stearates, linoleates, linolenates, caprates and arachidonates, or mixtures thereof, for instance mixed oleo-palmitates, oleo-stearates or palmito-stearates.

In at least one embodiment, mono- and diesters, such as sucrose, glucose or methylglucose mono- or dioleates, stearates, behenates, oleopalmitates, linoleates, linolenates or oleostearates may be used.

Mention may be made, for example, of the product sold under the name Glucate® DO by the company Amerchol, which is a methylglucose dioleate.

Mention may also be made, as examples of esters or of mixtures of esters of sugar and of fatty acid, of:

the products sold under the names F160, F140, F110, F90, F70 and SL40 by the company Crodesta, respectively denoting sucrose palmito-stearates formed from 73% monoester and 27% diester and triester, 61% monoester and 39% diester, triester and tetraester, 52% monoester and 48% diester, triester and tetraester, 45% monoester and 55% diester, triester and tetraester, and 39% monoester and 61% diester, triester and tetraester, and sucrose monolaurate;

the products sold under the name Ryoto Sugar Esters, for example referenced B370 and corresponding to sucrose behenate formed from 20% monoester and 80% di-triester-polyester;

sucrose mono-di-palmito-stearate sold by the company Goldschmidt under the name Tegosoft® PSE.

As regards the cyclic ethers and cyclic esters, the ones that are suitable include γ-butyrolactone, dimethyl isosorbide or diisopropyl isosorbide.

Silicone oils may also be used as the inert organic liquid phase.

For example, the silicone oils that are suitable are liquid, non-volatile silicone fluids with a viscosity of less than or equal to 10 000 mPa·s at 25° C., the viscosity of the silicones being measured according to ASTM standard 445 Appendix C.

Silicone oils are defined in greater detail in Walter Noll's “Chemistry and Technology of Silicones” (1968)—Academic Press.

Among the silicone oils that may be used according to the present disclosure, examples that may be mentioned include the silicone oils sold under the names DC-200 Fluid-5 mPa·s, DC-200 Fluid-20 mPa·s, DC-200 Fluid-350 mPa·s, DC-200 Fluid-1000 mPa·s and DC-200 Fluid-10 000 mPa·s by the company Dow Corning.

Mineral oils may also be used as inert organic liquid phase, for instance liquid paraffin.

Plant oils may also be suitable for use, especially avocado oil, olive oil or liquid jojoba wax.

In at least one embodiment, the inert organic liquid phase is chosen from the group formed by the polydecenes of formula C₁₀ₙH_{[(20n)+2]} in which n ranges from 3 to 9, such as from 3 to 7, esters of fatty alcohols or of fatty acids, and mixtures thereof.

According to one embodiment of the present disclosure, the content of inert organic liquid phase ranges from 5% to 60% by weight, such as from 10% to 50% by weight and for example from 15% to 45% by weight relative to the weight of the anhydrous paste.

According to one embodiment of the present disclosure, the composition in accordance with the present disclosure is anhydrous.

In the context of the present disclosure, a composition is anhydrous when it has a water content of less than 1% by weight, such as less than 0.5% by weight, relative to the total weight of the composition.

According to another embodiment of the present disclosure, the composition in accordance with the present disclosure also comprises hydrogen peroxide.

The pH of the composition containing hydrogen peroxide in accordance with the present disclosure generally ranges from 3 to 11, such as from 7 to 11.

The composition in accordance with the present disclosure may also comprise various additives conventionally used in cosmetics.

The composition in accordance with the present disclosure may thus comprise mineral or organic thickeners, such as associative or non-associative, anionic, cationic, nonionic or amphoteric thickening polymers, fillers such as clays, binders such as vinylpyrrolidone, lubricants, for instance polyol stearates or alkali metal or alkaline-earth metal stearates, hydrophilic or hydrophobic silicas, pigments, dyes other than those of the present disclosure, matting agents, for instance titanium oxides, or alternatively anionic, nonionic, cationic, amphoteric or zwitterionic surfactants, antioxidants, penetrants, sequestrants, buffers, dispersants, film-forming agents, preserving agents, opacifiers, vitamins, fragrances, anionic, cationic, nonionic, amphoteric or zwitterionic polymers, ceramides, and conditioning agents, for instance volatile or non-volatile, modified or unmodified silicones.

When the composition in accordance with the present disclosure comprises hydrogen peroxide, it may also comprise agents for controlling the release of oxygen, such as magnesium carbonate or oxide.

The additives and agents for controlling the release of oxygen as defined above may be present in an amount for each ranging from 0.01% to 40% by weight, such as from 0.1% to 30% by weight, relative to the total weight of the composition.

Needless to say, a person skilled in the art will take care to select this or these optional additional compound(s) such that the advantageous properties intrinsically associated with the composition in accordance with the invention are not, or are not substantially, adversely affected by the envisaged addition(s).

The process of bleaching and of simultaneous dyeing in accordance with the present invention comprises applying to keratin fibers a composition containing hydrogen peroxide in accordance with the disclosure as defined above.

A subject of the present disclosure is also a multi-compartment device, comprising at least two compositions, the mixing of which leads to a composition containing hydrogen peroxide as defined above.

According to one embodiment of the present disclosure, the device disclosed herein comprises a first compartment that contains a composition (A) comprising, in a suitable dyeing medium, at least one dye as defined above, a second compartment that contains an anhydrous composition (B) comprising at least one peroxygenated salt and at least one alkaline agent as defined above, and a third compartment that contains an aqueous hydrogen peroxide composition (E).

According to another embodiment of the invention, the device disclosed herein comprises a first compartment that contains an anhydrous composition (C) comprising at least one dye as defined above, at least one peroxygenated salt and at least one alkaline agent as defined above, and a second compartment that contains an aqueous hydrogen peroxide composition (E).

According to another embodiment of the present disclosure, the device disclosed herein comprises a first compartment that contains an anhydrous composition (B) comprising at least one peroxygenated salt and at least one alkaline agent as defined above, and a second compartment that contains a composition (D) comprising, in a suitable dyeing medium, at least one dye as defined above and hydrogen peroxide.

The suitable dyeing medium for compositions (A) and (D) generally comprises water or a mixture of water and of at least one organic solvent to dissolve the compounds that are not sufficiently water-soluble. Examples of organic solvents that may be mentioned include C₁-C₄ lower alkanols such as ethanol and isopropanol; glycerol; glycols and glycol ethers, for instance 2-butoxyethanol, propylene glycol, propylene glycol monomethyl ether, and also aromatic alcohols, for instance benzyl alcohol or phenoxyethanol, similar products and mixtures thereof.

The solvents may be present in an amount ranging from 1% to 40% by weight, such as from 5% to 30% by weight, relative to the total weight of the dye composition.

The composition (A), also known as the “booster”, may be formulated at acidic, neutral or alkaline pH, the pH possibly ranging from 3 to 12, such as from 4 to 11.

In at least one embodiment, the composition (D) has a pH of less than 7, the acidic pH ensuring the stability of the hydrogen peroxide in this composition.

The compositions (A) and (D) may be in various forms, such as in the form of liquids, creams or gels, or in any other form that is suitable for dyeing keratin fibers.

The anhydrous compositions (B) and (C) may be in the form of powder or paste. In this case, they also comprise an inert organic liquid phase as defined above.

In at least one embodiment, the aqueous hydrogen peroxide composition (E) has a pH of less than 7, the acidic pH ensuring the stability of the hydrogen peroxide in this composition.

The compositions (A), (B), (C), (D) and (E) may also contain various additives conventionally used in cosmetics, such as those described above.

The compositions (E) and (D) may also comprise agents for controlling the release of oxygen, as defined above.

The device in accordance with the present disclosure may be equipped with a means for applying the desired mixture to the hair, such as the devices described in French patent FR-2 586 913.

Using this device, it is possible to bleach and simultaneously dye keratin fibers by means of a process in accordance with the present disclosure as defined above.

A subject of the present disclosure is also the use, for bleaching and simultaneously dyeing keratin fibers, of a composition in accordance with the present disclosure as defined above.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible. Any numerical value, however, inherently contain certain errors necessarily resulting from the standard deviation found in their respective testing measurements.

The examples that follow serve to illustrate the invention without, however, being limiting in nature.

### EXAMPLE

Food Yellow 13 was dissolved in an aqueous-alcoholic solution (80/20) to a concentration of 3.5 g %. Booster A was thus obtained.

This solution was added just before use to a mixture of Platine Precision B powder comprising 51.5% of a mixture of sodium, potassium and magnesium persulfates in the presence of 4.2% of a mixture of sodium metasilicate and ammonium chloride with an oxidizing agent E consisting of a 40-volumes aqueous hydrogen peroxide composition. The proportions of the bleaching powder B/oxidizing agent E/booster A mixture were, respectively, 1/2/0.5.

A portion of this mixture was applied immediately to a 1 g lock of natural hair containing 90% white hairs, and also to a lock of 2.7 g of natural chestnut-brown hair.

The remainder of this mixture was applied 20 minutes later to a lock of 1 g of natural hair containing 90% white hairs and also to a lock of 2.7 g of natural chestnut-brown hair.

In all cases, the conditions were identical. The bath ratio was equal to 10.

After a leave-on time of 30 minutes, the locks were rinsed and then shampooed, rinsed again and dried.

The results are given in the table below.

TINTS OBTAINED AFTER APPLYING THE COMPOSITIONS
OF THE INVENTION

| | Natural hair containing | Natural chestnut-brown |
|---|---|---|
| | 90% white hairs | hair |
| Immediate application | Pale yellow | Coppery golden |
| of the mixture | | |
| Delayed application | Pale yellow | Coppery golden |
| of the mixture | | |

The natural hair containing 90% white hairs was used here to exacerbate any possible change in tint.

It was found that the same tint was obtained when the mixture was applied immediately and when its application was delayed.

These results show that the compositions in accordance with the invention are stable over time.

What is claimed is:

1. A composition for bleaching and simultaneously dyeing keratin fibers, comprising:
at least one dye chosen from quinoline, quinoline derivatives and the addition salts thereof;
at least one peroxygenated salt; and
at least one alkaline agent.

2. A composition according to claim 1, wherein the at least one dye is chosen from the compounds of formula (I) or (I′) below, and the addition salts thereof: 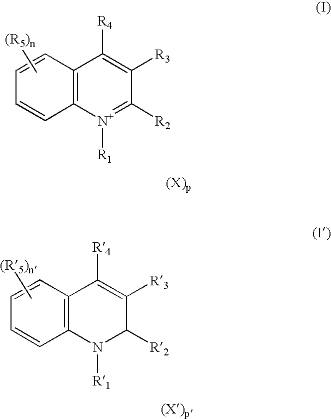
wherein:
R₁ and R₄ are chosen, independently of each other, from:
a hydrogen atom;
alkyl radicals; and
hydroxyalkyl radicals;
R₂ is chosen from:
a hydrogen atom; and
an alkyl radical;
R₃ is chosen from:
a hydrogen atom;
a saturated or unsaturated, 5- or 6-membered heterocyclic radical optionally fused with at least one ring chosen from aromatic or non-aromatic, heterocyclic or non-heterocyclic rings, the whole group comprising from 3 to 20 carbon atoms and from 1 to 4 hetero atoms, which may be substituted or unsubstituted, and optionally bearing a cationic charge; and
an alkyl radical;
R₅ is chosen from:
an alkyl radical;
an amino radical;
a monoalkylamino or dialkylamino radical;
a mono(hydroxyalkyl)amino or di(hydroxyalkyl)amino radical;
an N,N-(alkyl)(hydroxyalkyl)amino radical; and
a sulfonato radical;
n is an integer ranging from 0 to 4, with the proviso that when n is greater than or equal to 2, then the radicals R₅ may be identical or different;
X denotes a negatively charged organic or mineral atom or group of atoms to ensure the overall neutrality of the molecule;
p is an integer equal to 0 or 1;
R′₁, R′₃, R′₄, R′₅, n′, X′ and p′ have, respectively, the same definition as R₁, R₃, R₄, R₅, n, X and p;
R′₂ is chosen from:
a hydrogen atom;
an alkyl radical; and
a disubstituted methylidene radical of formula (II): 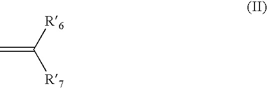
wherein R′₆ and R′₇ are chosen from, independently of each other, alkyl radicals; hydroxyalkyl radicals; or together form, with the carbon atom to which they are attached, a saturated or unsaturated 5- or 6-membered ring in which at least one carbon atom may be replaced with a carbonyl group, optionally fused with at least one ring chosen from aromatic or non-aromatic, heterocyclic or non-heterocyclic rings, the whole group possibly being unsubstituted or substituted with at least one radical chosen from an alkyl radical, an alkoxy radical and an amino radical.

3. A composition according to claim 2, in which R₁ and R₄ are chosen from, independently of each other, a hydrogen atom and an alkyl radical.

4. A composition according to claim 3, in which R₁ and R₄ are chosen from, independently of each other, a hydrogen atom; a methyl radical; and an ethyl radical.

5. A composition according to claim 2, in which R₂ is chosen from a hydrogen atom and an alkyl radical.

6. A composition according to claim 5, in which R₂ is chosen from a hydrogen atom and a methyl radical.

7. A composition according to claim 2, in which R₃ is chosen from a hydrogen atom; a substituted or unsubstituted quinolinium radical; and a substituted or unsubstituted 3H-indolium radical.

8. A composition according to claim 7, in which R₃ is chosen from a hydrogen atom; a 1-methylquinolinium radical; and a 1,3,3′-trimethyl-3H-indolium radical.

9. A composition according to claim 2, in which R₅ is chosen from a mono(alkyl)amino radical and a di(alkyl)amino radical.

10. A composition according to claim 9, in which R₅ is a dimethylamino radical.

11. A composition according to claim 2, in which n is equal to 0 or 1.

12. A composition according to claim 2, in which R′₁ and R′₄ are each a hydrogen atom.

13. A composition according to claim 2, in which R′₂ is a disubstituted methylidene radical of formula (II).

14. A composition according to claim 13, in which R′₂ is an indanedione radical.

15. A composition according to claim 2, in which R′₃ is a hydrogen atom.

16. A composition according to claim 2, in which R′₅ is chosen from a sulfonato radical and an alkyl radical.

17. A composition according to claim 16, in which R′₅ is chosen from a sulfonato radical and a methyl radical.

18. A composition according to claim 2, in which n′ ranges from 1 to 4.

19. A composition according to claim 18, in which at least one of the radicals R′₅ is a sulfonato radical.

20. A composition according to claim 19, in which at least one of the radicals R′₅ is a sulfonato radical in the form of the sodium salt.

21. A composition according to claim 1, in which the at least one dye is chosen from the following compounds: 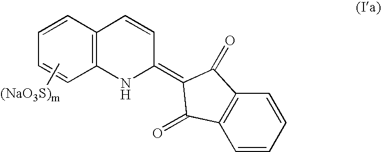
in which m is an integer ranging from 1 to 3; 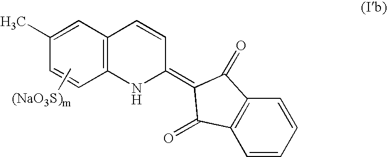
in which m is an integer ranging from 1 to 3;

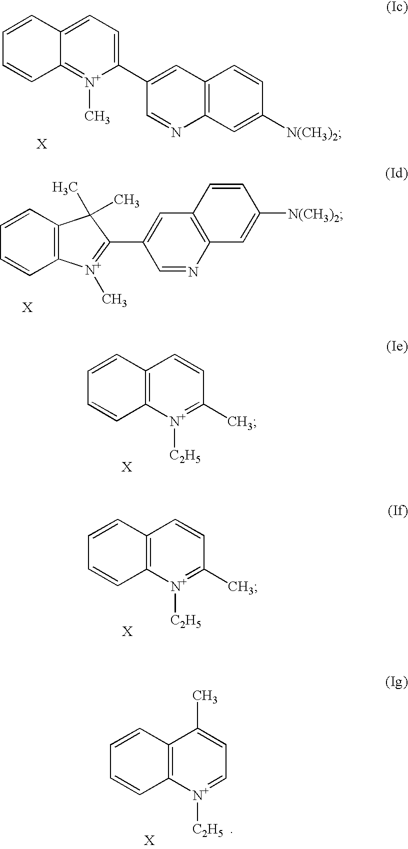

22. A composition according to claim 2, in which X is chosen from an alkylsulfonate and a halide.

23. A composition according to claim 21, in which X is chosen from an alkylsulfonate and a halide.

24. A composition according to claim 22, in which X is chosen from a methylsulfonate; a chloride; and an iodide.

25. A composition according to claim 23, in which X is chosen from a methylsulfonate; a chloride; and an iodide.

26. A composition according to claim 2, in which the at least one dye is chosen from the compounds of formula (I′), and the addition salts thereof.

27. A composition according to claim 21, in which the at least one dye is chosen from the compounds of formula (I′a); the compounds of formula (I′b); and the addition salts thereof.

28. A composition according to claim 27, in which the dye is Food Yellow 13.

29. A composition according to claim 1, in which the quinoline and/or quinoline derivatives and/or addition salts thereof are present in the composition in an amount ranging from 0.0001% to 10% by weight relative to the total weight of the composition.

30. A composition according to claim 1, in which the at least one peroxygenated salt is chosen from alkali metal and alkaline-earth metal persulfates, perborates, percarbonates and peroxides, and mixtures thereof.

31. A composition according to claim 30, in which the at least one peroxygenated salt is chosen from persulfates, and mixtures thereof.

32. A composition according to claim 29, in which the at least one peroxygenated salt is chosen from sodium persulfate, potassium persulfate and ammonium persulfate, and mixtures thereof.

33. A composition according to claim 1, in which the at least one peroxygenated salt is present in the composition in an amount ranging from 10% to 70% by weight relative to the total weight of the composition.

34. A composition according to claim 1, in which the at least one alkaline agent is chosen from urea, ammonium chloride, ammonium sulfate, ammonium phosphate, ammonium nitrate and alkali metal or alkaline-earth metal silicates, phosphates or carbonates, and mixtures thereof.

35. A composition according to claim 1, in which the at least one alkaline agent is present in an amount ranging from 0.01% to 40% by weight relative to the total weight of the composition.

36. A composition according to claim 1, further comprising at least one inert organic liquid phase.

37. A composition according to claim 35, in which the at least one inert organic liquid phase is chosen from the polydecenes of formula C₁₀ₙH_{[(20n)+2]} in which n ranges from 3 to 9, esters of fatty alcohols or of fatty acids, C₁₂-C₂₄ fatty acid esters or diesters of sugars, cyclic ethers or cyclic esters, silicone oils, mineral oils and plant oils, or mixtures thereof.

38. A composition according to claim 37, in which the at least one inert organic liquid phase is chosen from the polydecenes of formula C₁₀ₙH_{[(20n)+2]} in which n ranges from 3 to 9, and esters of fatty alcohols or of fatty acids, and mixtures thereof.

39. A composition according to claim 36, in which the at least one inert organic liquid phase is present in the composition in an amount ranging from 5% to 60% by weight relative to the total weight of the composition.

40. A composition according to claim 1, wherein said composition is anhydrous.

41. A composition according to claim 1, further comprising hydrogen peroxide.

42. A process for bleaching and simultaneously dyeing keratin fibers, comprising applying to said keratin fibers a composition comprising
at least one dye chosen from quinoline, quinoline derivatives and the addition salts thereof;
at least one peroxygenated salt; and
at least one alkaline agent.

43. A multi-compartment device, comprising at least two compositions, the mixing of which leads to a composition comprising:
at least one dye chosen from quinoline, quinoline derivatives and the addition salts thereof;
at least one peroxygenated salt; and
at least one alkaline agent.

44. A multi-compartment device comprising at least one first compartment containing at least one composition (A) comprising, in a suitable dyeing medium, at least one dye chosen from quinoline, quinoline derivatives and the addition salts thereof, at least one second compartment containing at least one anhydrous composition (B) comprising at least one peroxygenated salt, and at least one alkaline agent, and at least one third compartment containing at least one aqueous hydrogen peroxide composition (E).

45. A multi-compartment device comprising at least one first compartment containing at least one anhydrous composition (C) comprising at least one dye chosen from quinoline, quinoline derivatives and the addition salts thereof, at least one peroxygenated salt, and at least one alkaline agent, and at least one second compartment containing at least one aqueous hydrogen peroxide composition (E).

46. A multi-compartment device comprising at least one first compartment containing at least one anhydrous composition (B) comprising at least one peroxygenated salt, and at least one alkaline agent, and at least one second compartment containing at least one composition (D) comprising, in a suitable dyeing medium, at least one dye chosen from quinoline, quinoline derivatives and the addition salts thereof and hydrogen peroxide.
